SEQUENCE LISTING (22 sequences)

SEQ ID NO: 1 - protein, 108 aa, Artificial Sequence
  Amino acid sequence identified using molecular biology techniques.
DIQMTQSPSSLSASVGDRVTITCRASQDIYLNLDWYQQKPGKAPKLLINFGSELQSGVPSRFSGSGYGTDFTLTISSLQPEDFATYYCQPSFYFPYTFGQGTKVEIKR

SEQ ID NO: 2 - protein, 116 aa, Artificial Sequence
  Amino acid sequence identified using molecular biology techniques.
EVQLLESGGGLVQPGGSLRLSCAASGFTFRNFGMGWVRQAPGKGLEWVSWIISSGTETYYADSVKGRFTISRDNSKNTLYLQMNSLRAEDTAVYYCAKSLGRFDYWGQGTLVTVSS

SEQ ID NO: 3 - protein, 119 aa, Artificial Sequence
  Amino acid sequence identified using molecular biology techniques.
EVQLLESGGGLVQPGGSLRLSCAASGFTFAHETMVWVRQAPGKGLEWVSHIPPDGQDPFYADSVKGRFTISRDNSKNTLYLQMNSLRAEDTAVYHCALLPKRGPWFDYWGQGTLVTVSS

SEQ ID NO: 4 - protein, 119 aa, Artificial Sequence
  Amino acid sequence identified using molecular biology techniques.
EVQLLESGGGLVQPGGSLRLSCAASGFTFSEGTMWWVRQAPGKGLEWVSAILAAGSNTYYADSVKGRFTISRDNSKNTLYLQMNSLRAEDTAVYYCAKKRQERDGFDYWGQGTLVTVSS

SEQ ID NO: 5 - protein, 120 aa, Artificial Sequence
  Amino acid sequence identified using molecular biology techniques.
EVQLLESGGGLVQPGGSLRLSCAASGSTFTDDRMWWVRQAPGKGLEWVSAIQPDGHTTYYADSVKGRFTISRDNSKNTLYLQMNSLRAEDTAVYYCAEQDVKGSSSFDYWGQGTLVTVSS

SEQ ID NO: 6 - protein, 119 aa, Artificial Sequence; identical to SEQ ID NO: 3
  Amino acid sequence identified using molecular biology techniques.

SEQ ID NO: 7 - protein, 233 aa, Homo sapien
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 8 - protein, 189 aa, Homo sapien
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 9 - protein, 525 aa, Homo sapien
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 10 - protein, 212 aa, Homo sapien
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 11 - protein, 146 aa, Homo sapien [sequence omitted]

SEQ ID NO: 12 - protein, 193 aa, Homo sapien
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 13 - protein, 159 aa, Homo sapien
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 14 - protein, 171 aa, Homo sapien
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 15 - protein, 207 aa, Homo sapien
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 16 - protein, 182 aa, Homo sapien
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 17 - protein, 164 aa, Homo sapien
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 18 - protein, 312 aa, Homo sapien
MAAGQNGHEEWVGSAYLFVESSLDKVVLSDAYAHPQQKVAVYRALQAALAESGGSPDVLQMLKIHRSDPQLIVQLRFCGRQPCGRFLRAYREGALRAALQRSLAAALAQHSVPLQLELRAGAERLDALLADEERCLSCILAQQPDRLRDEELAELEDALRNLKCGSGARGGDGEVASAPLQPPVPSLSEVKPPPPPPPAQTFLFQGQPVVNRPLSLKDQQTFARSVGLKWRKVGRSLQRGCRALRDPALDSLAYEYEREGLYEQAFQLLRRFVQAEGRRATLQRLVEALEENELTSLAEDLLGLTDPNGGLA

SEQ ID NO: 19 - protein, 107 aa, Homo sapien
  Amino acid sequence identified using molecular biology techniques.
MARATLSAAPSNPRLLRVALLLLLLVAASRRAAGAPLATELRCQCLQTLQGIHLKNIQSVKVKSPGPHCAQTEVIATLKNGQKACLNPASPMVKKIIEKMLKNGKSN

SEQ ID NO: 20 - protein, 114 aa, Homo sapien
MSLLSSRAARVPGPSSSLCALLVLLLLLTQPGPIASAGPAAAVLRELRCVCLQTTQGVHPKMISNLQVFAIGPQCSKVEVVASLKNGKEICLDPEAPFLKKVIQKILDGGNKEN

SEQ ID NO: 21 - protein, 107 aa, Homo sapien
MARAALSAAPSNPRLLRVALLLLLLVAAGRRAAGASVATELRCQCLQTLQGIHPKNIQSVNVKSPGPHCAQTEVIATLKNGRKACLNPASPIVKKIIEKMLNSDKSN

SEQ ID NO: 22 - protein, 107 aa, Homo sapien
MAHATLSAAPSNPRLLRVALLLLLLVAASRRAAGASVVTELRCQCLQTLQGIHLKNIQSVNVRSPGPHCAQTEVIATLKNGKKACLNPASPMVQKIIEKILNKGSTN

(19) United States
(12) Patent Application Publication
(10) Pub. No.: US 2015/0306058 A1
(43) Pub. Date: Oct. 29, 2015

(54) COMPOSITION COMPRISING A SINGLE VARIABLE DOMAIN AND CAMOSTAT MESYLATE (CM)

(71) Applicant: Glaxo Group Limited, Brentford, Middlesex (GB)

(72) Inventors: Sean Matthew CLEVELAND, Stevenage (GB); Stefan SALOMON, Stevenage (GB); Cassandra VAN KRINKS, Stevenage (GB)

(21) Appl. No.: 14/422,706

(22) PCT Filed: Aug. 21, 2013
(86) PCT No.: PCT/IB2013/001814

§ 371 Date: Feb. 20, 2015

Related U.S. Application Data
(60) Provisional application No. 61/691,443, filed on Aug. 21, 2012.

(51) Int. Cl. A61K 31/245; A61K 38/20; A61K 38/17; A61K 38/19

(52) U.S. Cl. CPC A61K 31/245; A61K 38/191; A61K 38/20; A61K 38/204; A61K 38/2086; A61K 38/177

(57) ABSTRACT

The present disclosure provides a means of stabilising a single variable domain, in particular in protease-rich environments such as the stomach and intestine. A composition, in particular a pharmaceutical composition, comprising a single variable domain and camostat mesylate is provided, together with uses of said composition as a medicament and in methods of treatment. Compositions of the disclosure are particularly useful in the topical treatment of gastrointestinal conditions, such as Crohn's Disease or ulcerative colitis, or for direct activity in the gut mucosal immune system.

### BACKGROUND OF THE DISCLOSURE

The vast majority of biopharmaceuticals, particularly therapeutic antibodies and their fragments, are administered by the parenteral route, e.g. by intravenous or subcutaneous injection. These routes of administration can often be inconvenient and painful which reduces patient compliance, particularly when multiple injections per day are required. They can also be costly to health care providers, in terms of staff hours, storage and equipment.

Oral administration of biopharmaceuticals would overcome many of these drawbacks but has its own challenges. In particular, such molecules are subject to proteolytic degradation in the protease-rich environment of the stomach and intestine.

Importantly, there is a need for oral therapeutics that treat diseases of the gastrointestinal (GI) tract. In particular there is a need for lower doses of drug to be used to lower the risk of systemic toxicity.

Thus, there is a strong need to stabilise proteins in order to allow them to withstand the protease-rich environment of the gastrointestinal tract thus enabling the successful oral administration of biopharmaceuticals.

### SUMMARY OF THE DISCLOSURE

The disclosure provides a composition, optionally a pharmaceutical composition, comprising camostat mesylate and a single variable domain.

A composition of the disclosure for use as a medicament is provided. The use of a composition of the disclosure for the manufacture of a medicament is also provided. In particular the composition is to be administered orally.

The disclosure provides a method of treating a gastrointestinal condition comprising the step of administering, optionally orally, a composition of the disclosure to a patient in need thereof.

The disclosure further provides a method of stabilising a single variable domain in a protease-rich solution comprising formulating the single variable domain in a composition comprising camostat mesylate prior to exposing the composition to a protease-rich solution.

### BRIEF DESCRIPTION OF THE FIGURES

FIG. 1 shows the half-life of a panel of dAbs™ with different transition midpoints (Tm), upon incubation in simulated intestinal fluid (SIF).

FIG. 2 shows the half-life of a panel of high Tm dAbs™, upon incubation in SIF.

FIG. 3 shows the half-life of a panel of dAbs™ with different transition midpoints (Tm), upon incubation in simulated intestinal fluid (SIF), in the presence and absence of CM.

FIG. 4 shows the half-life of a panel of high Tm dAbs™, upon incubation in SIF, in the presence and absence of CM.

FIG. 5 shows the half-life of two dAbs™ with identical predicted trypsin cleavage sites but differing Tm. The dAbs™ were incubated with trypsin, in the presence and absence of CM.

FIG. 6 shows the amount of the dAb™ DOM101 recovered from gut tissue at various time-points after intra-duodenal administration in the absence (a), and presence (b) of CM. Results are expressed as nanograms per gram of tissue.

FIG. 7 shows the amount of the dAb™ DOM101 recovered from the large intestine after intra-colonic administration in the presence and absence of CM. Results are expressed as nanograms per gram of tissue.

### DETAILED DESCRIPTION

The present disclosure provides a solution to the problems discussed above. The present disclosure provides a means of stabilising single variable domains. A composition, in particular a pharmaceutical composition, comprising a single variable domain and camostat mesylate is provided, together with uses of said composition as a medicament and in methods of treatment. The examples herein show that camostat mesylate (CM) can be used to stabilise single variable domains (e.g. domain Antibodies™ or dAbs™) both in fasted simulated intestinal fluid and in the small and large intestine, and are thus supportive of the use of CM for the oral delivery of biopharmaceuticals for topical treatment of GI conditions, such as Crohn's Disease or ulcerative colitis or for direct activity in the gut mucosal immune system.

The chemical name for camostat mesylate (CAS No: 59721-29-8) is 4-[[4-[(Aminoiminomethyl)amino]benzoyl]oxy]benzeneacetic acid 2-(dimethylamino)-2-oxoethyl ester methanesulfonate and it can be obtained, for example, from Sequoia Research Products. Camostat mesylate (CM) is an orally active serine protease inhibitor, which is licensed in Japan and Korea for the treatment of pancreatitis and post-operative reflux oesophagitis (Foipan Product information sheet; Takasugi et al., Digestion 1982, 24:36-41; Kono et al., Am J Surg. 2005 September, 190(3): 412-7). CM has a broad spectrum of inhibition, including trypsin, thrombin, kallikrein and plasmin (Tamura et al., 1977, Biochimica et Biophysica Acta 484, 417-422). The metabolism of CM within the gut is not clear, however the metabolite of CM, GBPA, is itself active (Beckh et al., Res Exp Med, 1987, 187: 401-406).

The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as fragments of variable domains which retain at least the binding activity and specificity of the full-length domain. A single variable domain is capable of binding an antigen or epitope independently of a different variable region or domain. A “domain Antibody™” or “dAb™)” may be considered the same as a “single variable domain”. A single variable domain may be a human single variable domain, but also includes single variable domains from other species such as rodent (for example, as disclosed in WO 00/29004), nurse shark and Camelid VHH dAbs™. Camelid VHH are immunoglobulin single variable domains that are derived from species including camel, llama, alpaca, dromedary, and guanaco, which produce heavy chain antibodies naturally devoid of light chains. Such VHH domains may be humanised according to standard techniques available in the art, and such domains are considered to be “single variable domains”. As used herein VH includes camelid VHH domains.

An anti-target single variable domain, e.g. an anti-TNFα single variable domain, refers to a single variable domain which binds to said target, e.g. TNFα. The target may be any suitable target. In an embodiment a single variable domain of the disclosure targets any one of the following: TNFα, IL-23, LAG-3, IL-6, IL-13, IL-18, TSLP, CD3 or a receptor of any one of the foregoing, e.g. a TNFα receptor, such as TNFRαRI or TNFRαRII, an IL-23 receptor, a LAG-3 receptor, an IL-6 receptor, an IL-13 receptor, an IL-18 receptor, a TSLP receptor, or a CD3 receptor. In an embodiment a single variable domain of the disclosure targets a chemokine or a chemokine receptor e.g. a glutamic acid-leucine-arginine receptor i.e. an ELR receptor such as one comprising the amino acid sequence shown in SEQ ID NOs: 12 and 19-22.

Affinity is the strength of binding of one molecule, e.g. a single variable domain of the disclosure, to another, e.g. its target, at a single binding site. The binding affinity of a single variable domain to its target may be determined by equilibrium methods (e.g. enzyme-linked immunoabsorbent assay (ELISA) or radioimmunoassay (RIA)), or kinetics (e.g. BIACORE™ analysis).

In an embodiment, the equilibrium dissociation constant (KD) of the single variable domain-target interaction is 100 nM or less, 10 nM or less, 2 nM or less or 1 nM or less. Alternatively the KD may be between 5 and 10 nM; or between 1 and 2 nM. The KD may be between 1 pM and 500 pM; or between 500 pM and 1 nM. A skilled person will appreciate that the smaller the KD numerical value, the stronger the binding. The reciprocal of KD (i.e. 1/KD) is the equilibrium association constant (KA) having units M⁻¹. A skilled person will appreciate that the larger the KA numerical value, the stronger the binding.

The dissociation rate constant (kd) or “off-rate” describes the stability of the single variable domain-target complex, i.e. the fraction of complexes that decay per second. For example, a kd of 0.01 s⁻¹ equates to 1% of the complexes decaying per second. In an embodiment, the dissociation rate constant (kd) is 1×10⁻³ s⁻¹ or less, 1×10 s⁻¹ or less, 1×10⁻⁵ s⁻¹ or less, or 1×10⁻⁶ s⁻¹ or less. The kd may be between 1×10⁻⁵ s⁻¹ and 1×10⁻⁴ s⁻¹; or between 1×10⁻⁴ s⁻¹ and 1×10⁻³ s⁻¹.

The term “neutralises” as used throughout the present specification means that the biological activity of target is reduced in the presence of a single variable domain as described herein in comparison to the activity of target in the absence of the single variable domain, in vitro or in vivo. Neutralisation may be due to one or more of blocking the target binding to its receptor, preventing target from activating its receptor, down regulating the target or its receptor, or affecting effector functionality. In an embodiment, a single variable domain of the disclosure neutralises its target.

“Transition midpoint” or “Tm” is the temperature where 50% of the single variable domain is in its native conformation and the other 50% is denatured. In an embodiment, the single variable domain has a high Tm. In particular the Tm is greater than or equal to about 66° C. The thermal stability of a single variable domain, including the Tm, may be determined using Differential Scanning calorimetry (DSC).

“Oral administration” as used herein refers to the administration of compositions as disclosed herein by mouth. Compositions of the disclosure are typically swallowed and travel into the gastrointestinal (GI) tract where they act. Small amounts may be absorbed across the intestinal mucosa into the circulation for systemic action. Absorption may begin in the mouth (buccal cavity) and stomach, but usually occurs in the small intestine.

The “gastrointestinal (GI) tract” includes the upper GI tract: mouth, pharynx, oesophagus and stomach; and the lower GI tract: small intestine, duodenum, jejunum, ileum, large intestine (caecum, colon—including the ascending colon, transverse colon, descending colon and sigmoid flexure), rectum and anus; as well as the gall bladder, liver and pancreas. Compositions of the disclosure may target any one or more of the aforementioned regions of the GI tract. In an embodiment, compositions target the small intestine. In an embodiment, compositions target the large intestine.

Pharmaceutical compositions disclosed herein may be for the treatment of any one or more of the human diseases described herein. In one embodiment, the pharmaceutical composition comprises a single variable domain optionally in combination with one or more pharmaceutically acceptable carriers and/or excipients.

Such compositions comprise a pharmaceutically acceptable carrier as known and called for by acceptable pharmaceutical practice, see e.g. Remingtons Pharmaceutical Sciences, 16th edition (1980) Mack Publishing Co. Methods for the preparation of such pharmaceutical compositions are well known to those skilled in the art.

In an embodiment, pharmaceutical compositions of the disclosure are to be administered orally. A variety of dosage forms are contemplated, including liquids (solutions, suspensions (aqueous or oily), and emulsions), semi-solids (pastes), films and solids (tablets, lozenges, capsules, powders, crystals and granules).

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol.

Pharmaceutical compositions, in particular solid compositions such as tablets and capsules, may be enterically coated. Materials used for enteric coatings include fatty acids, waxes, shellac, plastics, and plant fibres. Suitable enteric coatings are disclosed in the EURDAGIT® Application Guidelines (11^{th} edition, 09/2009).

Effective doses and treatment regimes for administering the single variable domain may be dependent on factors such as the age, weight and health status of the patient and disease to be treated. Such factors are within the purview of the attending physician. Guidance in selecting appropriate doses may be found in e.g. Smith et al (1977) Antibodies in human diagnosis and therapy, Raven Press, New York.

The ratio of single variable domain to camostat mesylate in compositions of the disclosure may be about 1:0.1; 1:1; 1:10, 1:25, 1:50, or 1:100. In an embodiment the ratio of single variable domain to camostat mesylate in compositions of the disclosure is about 1:100. In an embodiment the ratio of single variable domain to camostat mesylate in compositions of the disclosure is about 1:10.

The pharmaceutical composition may comprise a kit of parts of the single variable domain together with other medicaments, optionally with instructions for use. For convenience, the kit may comprise the reagents in predetermined amounts with instructions for use.

The disclosure provides methods of treating diseases disclosed herein comprising the step of administering compositions of the disclosure to a patient in need thereof.

The present disclosure also provides the use of compositions of the disclosure as described herein in the manufacture of a medicament for the treatment of the diseases and disorders listed herein. Diseases and disorders which may be treated by compositions of the disclosure include gastrointestinal disorders.

A “gastrointestinal disorder” is a disorder affecting the GI tract and includes enteritis, proctitis, inflammatory bowel disease (IBD) including Crohn's disease, colitis including ulcerative colitis, celiac disease, Behet's syndrome and oral mucositis. In an embodiment the gastrointestinal disorder is IBD. In an embodiment the gastrointestinal disorder is Crohn's disease. In an embodiment the gastrointestinal disorder is ulcerative colitis.

Any other disease which may be treated by targeting the GI tract is encompassed within diseases to be treated by the methods of the disclosure. For example, a single variable domain of the disclosure which binds to a target within the GI tract may result in effects which go beyond the GI tract and result in the treatment of a systemic disease.

The terms “individual”, “subject” and “patient” are used herein interchangeably. The subject is typically a human. The subject may also be a mammal, such as a mouse, rat or primate (e.g. a marmoset or monkey). The subject can be a non-human animal.

Treatment can be therapeutic, prophylactic or preventative. The subject will be one who is in need thereof. Those in need of treatment may include individuals already suffering from a particular medical disease in addition to those who may develop the disease in the future. A therapeutically effective amount of the single variable domain described herein is an amount effective to ameliorate or reduce one or more symptoms of, or to prevent or cure, the disease.

A method of stabilising a single variable domain in a protease-rich solution is provided. The method comprises formulating the single variable domain in a composition comprising camostat mesylate prior to exposing the composition to a protease-rich solution.

A “protease-rich” solution is a solution comprising a protease, in particular a protease found in the GI tract, for example in a physiological amount. A protease is an enzyme that conducts proteolysis by hydrolysing one or more peptide bonds in a polypeptide chain. A physiological amount of trypsin inter-digestively in a human is 20-50 U/ml. A physiological amount of trypsin early postprandially in a human is 60-100 U/ml. A physiological amount of trypsin late postprandially in a human is 500-1500 U/ml (McConnell et al., International Journal of Pharmaceutics 364: 213-226 (2008)). In an embodiment, the trypsin amount in a protease-rich solution may be any of the aforementioned ranges. In an embodiment, the protease-rich solution comprises trypsin in an amount greater than any one of the following amounts: 20 U/ml, 30 U/ml, 40 U/ml, 50 U/ml, 60 U/ml, 70 U/ml, 80 U/ml, 90 U/ml, 100 U/ml, 200 U/ml, 300 U/ml, 400 U/ml, 500 U/ml, 600 U/ml, 700 U/ml, 800 U/ml, 900 U/ml, 1000 U/ml, 1100 U/ml, 1200 U/ml, 1300 U/ml, 1400 U/ml or 1500 U/ml. In an embodiment, the protease-rich solution may further comprise chymotrypsin and/or pancreatin. In an embodiment, the protease-rich solution comprises trypsin, chymotrypsin and/or pancreatin. In an embodiment, the protease-rich solution is simulated intestinal fluid (SIF). SIF comprises bile, pancreatin and trypsin. SIF may also comprise sodium chloride, potassium chloride and calcium chloride. In an embodiment the SIF is as described in Example, e.g. comprising the proteases in the amounts specified in Example 1.

Within this specification the disclosure has been described, with reference to embodiments, in a way which enables a clear and concise specification to be written. It is intended and should be appreciated that embodiments may be variously combined or separated without parting from the disclosure.

### EXAMPLES

### Example 1

### Intrinsic Stability of a Panel of Domain Antibodies™ in Simulated Intestinal Fluid (SIF)

Simulated intestinal fluid (SIF) was formulated based on a recipe used in the TNO-TIM™ gut model system, but with the volume substantially scaled down, as detailed below.

Simulated Intestinal Fluid (SIF) Preparation:

Bile solution was prepared by gently adding, with continuous stirring, 2.0 g (+/−0.02 g) of bile powder into 250 g (+/−5 g) of purified water until a clear solution was obtained.

Pancreatin solution was prepared by adding 2.1 g (+/−0.2 g) of pancreatin powder to 150 g (+/−3 g) of purified water. A stirrer was used and care was taken to minimise foaming. Once a homogenous mixture was obtained, the solution was centrifuged at 3500 rpm for 20 minutes and the supernatant was then stored on ice.

Small intestine electrolyte solution (SIES) 25% (concentrated) was produced by adding purified water to 250 g (+/−5 g) sodium chloride, 30 g (+/−0.5 g) potassium chloride, and 15 g (+/−0.3 g) calcium chloride dehydrate to make a total of 2174 g. Once the salts had dissolved the pH was adjusted to pH7.0 (+/−0.5) with 1M sodium hydroxide.

SIES dilute was then prepared using 43.5 (+/−1 g) SIES concentrate added to purified water to a total weight of 1000 g.

Trypsin solution was prepared by dissolving 200 mg (+/−5 mg) of trypsin in 100 g (+/−2 g) of SIES dilute. This solution was then pipetted into 1.5 ml eppendorf tubes (1 ml per tube) and frozen at −20° C.

The SIF was then prepared by mixing 25 g (+/−0.3 g) of bile solution, 12.5 g (+/−0.3 g) pancreatin solution and 12.5 g (+/−0.5 g) of SIES dilute (ratio 2:1:1 bile/pancreatin/SIES dilute). 1 ml of trypsin solution was then added prior to the immediate use of the solution.

Domain Antibody™ Preparation

Domain Antibodies™ (dAbs™) under investigation were concentrated to approximately 20 mg/ml using Vivaspin™ 500 3kD MWCO columns. Columns were pre-rinsed with PBS prior to use to maximise sample recovery. Concentration was confirmed by Nanodrop™ using the molar extinction co-efficient and molecular weight option.

Reaction Assembly

Incubations of dAb™ in SIF were carried out in a final volume of 250 μl. The volume of dAb™ spiked into the mixture provided a final concentration of 1 mg/ml.

A 25 μl aliquot was immediately removed and stored on dry ice (0 hour time point). Reaction mixtures were incubated at 37° C. with shaking (100 rpm). Subsequent 25 μl aliquots were removed at: 0.25 hours, 0.5 hours, 1 hour, 2 hours, 4 hours, 6 hours and overnight. Samples were snap frozen on dry ice and stored at −80° C. prior to analysis.

SDS-PAGE Analysis

The amount of dAb™ remaining in the SIF at various time-points was measured by SDS-PAGE and densitometry. Briefly, sample was diluted 1/10 in a water and sample loading buffer mixture, and heated to 80° C. for 5 min. Samples were quickly chilled, then 10 μl loaded into a 4-12% Novex™ bis-tris gel along with a prepared standard (dAb™) in water) and a molecular weight marker. The gel was run at 150V constant in 1×MES buffer for 45 minutes, and the protein bands visualised by staining with Instant Blue™ overnight. Densitometry of the resulting bands was performed using the Odyssey Li-Cor™ gel imaging system and the amount of dAb™ present calculated relative to the density of the 0 h time-point band (starting amount). An exponential curve of time vs. percentage of starting amount of dAb™ was prepared, and the time at which 50% of the starting amount of dAb™ was present was taken to be the half-life.

Using the methods above, a panel of dAbs™ with varying transition midpoints (Tm), as shown in table 1, were incubated in SIF and analysed by SDS-PAGE and densitometry. For these Examples, high Tm dAb™ refers to a dAb™ with a Tm of 66° C., and low Tm dAb™ refers to a dAb™ with a Tm of 56° C.

TABLE 1
Panel of dAbs⁽ ™⁾ with varying Tm

| dAb⁽ ™⁾ | Tm (° C.) | Framework |
|---|---|---|
| DOM1 (SEQ ID NO: 1) | 55.0 | V_{κ} |
| DOM2 (SEQ ID NO: 2) | 55.9 | Vh |
| DOM3 (SEQ ID NO: 3) | 65 | Vh |
| DOM4 | 72.8 | V_{κ} |
| DOM5 | 49 | Vh |
| DOM6 (SEQ ID NO: 4) | 55.8 | Vh |
| DOM7 (SEQ ID NO: 5) | 50.6 | Vh |

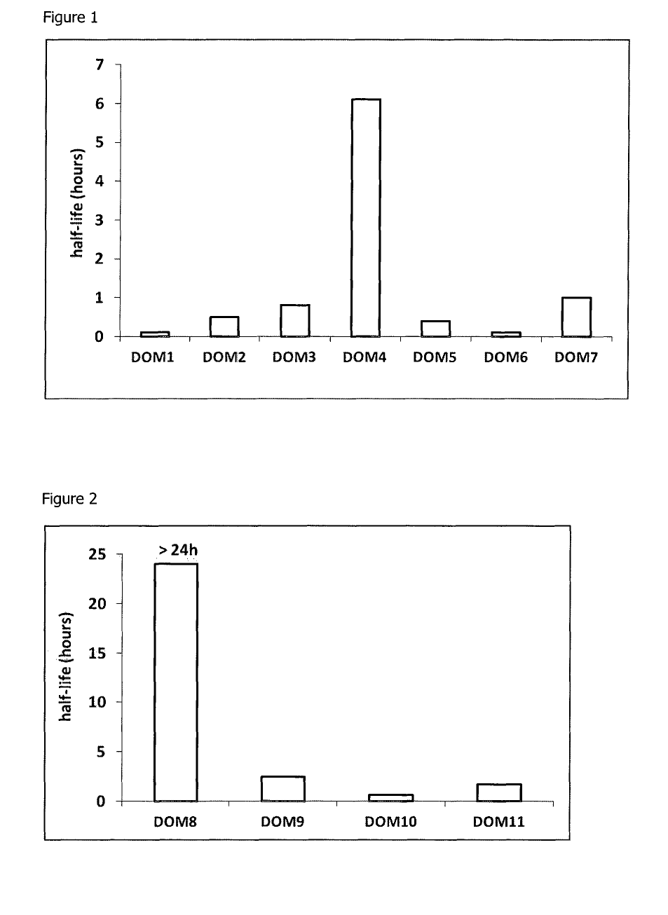

The results are shown in FIG. 1. This graph is a combination of SIF studies performed on three separate days. DOM4, the dAb™ with the highest Tm, was clearly much more stable that the other dAbs™ under investigation. To see if this was a trend, four further high Tm dAbs™, as shown in table 2, were studied using the methods above.

TABLE 2
Panel of high Tm dAbs⁽ ™⁾

| dAb⁽ ™⁾ | Tm (° C.) | Framework |
|---|---|---|
| DOM8 | 66.2 | V_{κ} |
| DOM9 | 74.3 | Vh |
| DOM10 | 73.7 | Vh |
| DOM11 | 68.2 | V_{κ} |

The results for the panel of high Tm dAbs™ are shown in FIG. 2.

One other dAb™, DOM8, was extremely stable in SIF. The other three dAbs™ were not as stable. However, four of the five high Tm dAbs™ tested were more stable than dAbs™ with a Tm below 66° C. The two most stable dAbs™ (DOM4 and DOM8) both had a Vκ framework. However, DOM11 also had a Vκ framework but was much less stable, so the framework may not be so important for stability. DOM11 was incubated in SIF on a different occasion to the other three high Tm dAbs™ tested here.

### Example 2

### Stabilisation of Domain Antibodies™ In Vitro Using Camostat Mesylate

The panel of dAbs™ studied in Example 1 were also incubated in SIF in the presence of camostat mesylate (CM, Sequoia Research Products), to determine whether inhibition of proteases would help to stabilise the dAbs™ further. CM was added to the electrolyte solution stated above in the SIF preparation section at a concentration of 350 mg/ml (CM was highly concentrated but below point of saturation) and warmed to 50° C. to dissolve. CM was added to the SIF/dAb™ at a final concentration of 10 mg/ml. The time-points used and subsequent analysis was performed as in Example 1.

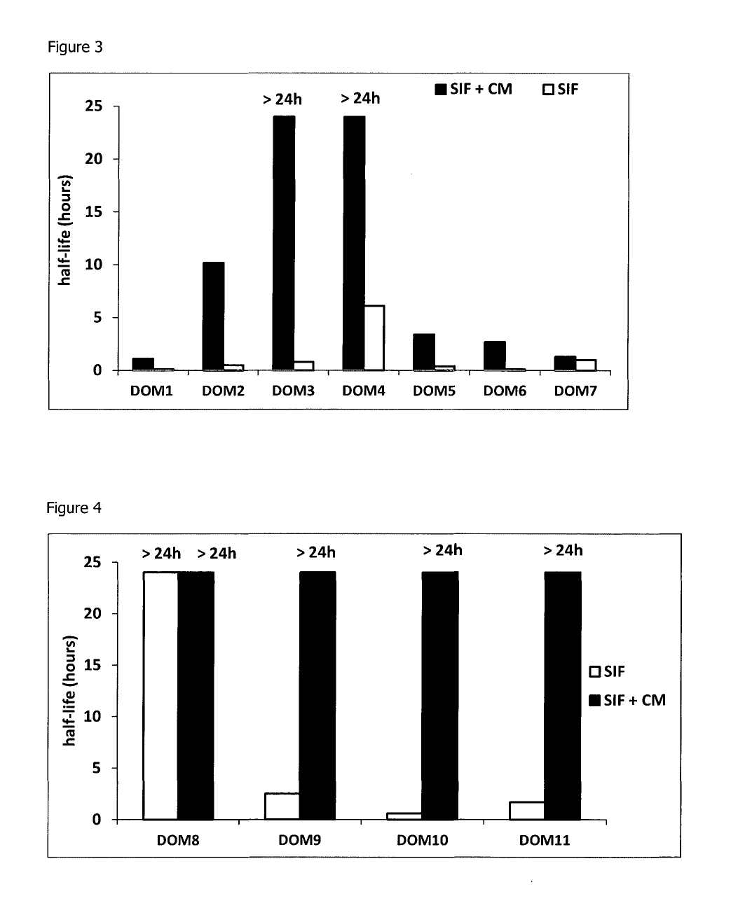

Results are shown alongside those from Example 1 for comparison in FIGS. 3 and 4. Addition of CM to the SIF/dAb mixture increased the half-life of all but one of the dAbs™ studied. The half-life extension was not the same for all molecules tested, suggesting that intrinsic properties of the dAbs™ contribute to their ability to be stabilised. In addition, the high Tm dAbs™, despite their variable half-lives, appear to be inherently more amenable to stabilisation with CM, as the half-life was extended to more than 24 hours for all the high Tm dAbs™ tested.

### Example 3

### Modelling of dAb™ Stability and Importance of Tm for the Inherent Stability of a Domain Antibody™

A perl script was written to scan protein sequences for the trypsin and chymotrypsin (present in pancreatin) cleavage sites. Half-life was then correlated with predicted cleavage sites, and with Tm.

A weak positive correlation was observed between Tm and half-life (Spearman, 0.58; Pearson, 0.31). However, a strong positive correlation was observed between Tm and half-life in the presence of CM using both correlation measures (Spearman, 0.78; Pearson, 0.90). This suggests that the higher the Tm, the more amenable the dAb™ to stabilisation with CM. No clear correlations were observed between predicted cleavage sites and half-life, in the presence or absence of CM.

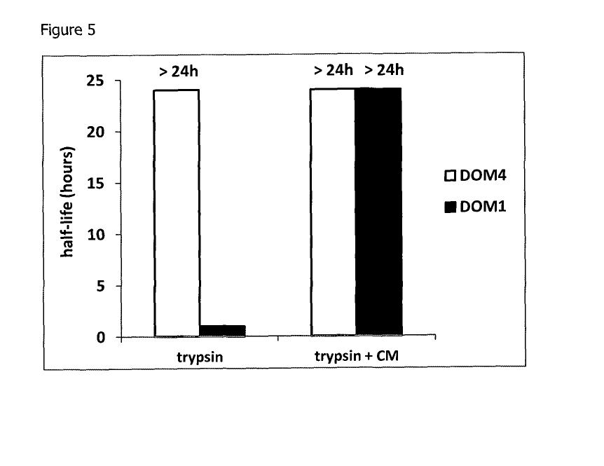

During the modelling process, two Vκ framework dAbs™ were observed to have identical predicted trypsin cleavage sites, but different half-lives in SIF and different Tm. These were DOM4 (half-life 6.1 hours, Tm 72.8° C.) and DOM1 (half-life 0.1 hours, Tm 55° C.). These two dAbs™ were incubated with trypsin, at the same concentration used in the SIF, but without bile salts or pancreatin. Any differences seen in half-life would then be due to Tm. CM was also added to the trypsin/dAb™ mixture. Half-life was calculated as before and results are shown in FIG. 5.

In the presence of trypsin alone, the half-life of the DOM4 was considerably longer than that of DOM1. In this instance, the difference in Tm likely accounted for the increased stability of the molecule.

### Example 4

### Use of Camostat Mesylate to Stabilise the TNFR1 Specific dAb™ DOM101 (SEQ ID NO:6) Administered Directly into the Duodenum of Fasted Han Wistar Rats

Han Wistar rats were dosed with 1 mg DOM101 in the presence or absence of 100 mg CM, to determine if CM preserved the dAb™ in the gastrointestinal tract. Rats were briefly anaesthetised by isoflurane anaesthetic and a midline abdominal incision made to facilitate location of the duodenum for direct intra-duodenal injection (500 μl) of the dose formulations. Following dosing, the abdominal incisions were closed and the rats allowed to recover prior to their return to study cages. Direct dosing into the duodenum bypassed the acidic conditions of the gastric juices in the stomach and allowed for direct analysis of pharmacokinetics in the intestinal tract.

Animals were culled at the following time-points: 0.5, 1.5, 3, 5, 7 and 18 hours (three animals per group).

Blood samples were taken and the intestinal tract dissected out and divided into its constituent parts: duodenum (×2), jejunum (×6), ileum, caecum, colon (×2), rectum.

Intestinal samples were homogenised using the GentleMACS™ Dissociator in lysis buffer containing detergent and protease inhibitors. Samples were screened for DOM101 using a TNFR1-specific MSD™ assay. In brief, MSD plates were coated with TNFR1-Fc. Plates were washed and blocked with bovine serum albumin. Tissue samples were diluted and added to the plate, along with a standard curve of dAb™, then incubated at room temperature to allow binding. Plates were washed and a sulfo-tag-conjugated anti-Vh antibody was added to the wells. After incubation, the plate was washed and incubated with MSD read buffer. The resulting electrochemiluminescence signal was read on a Sector Imager 6000.

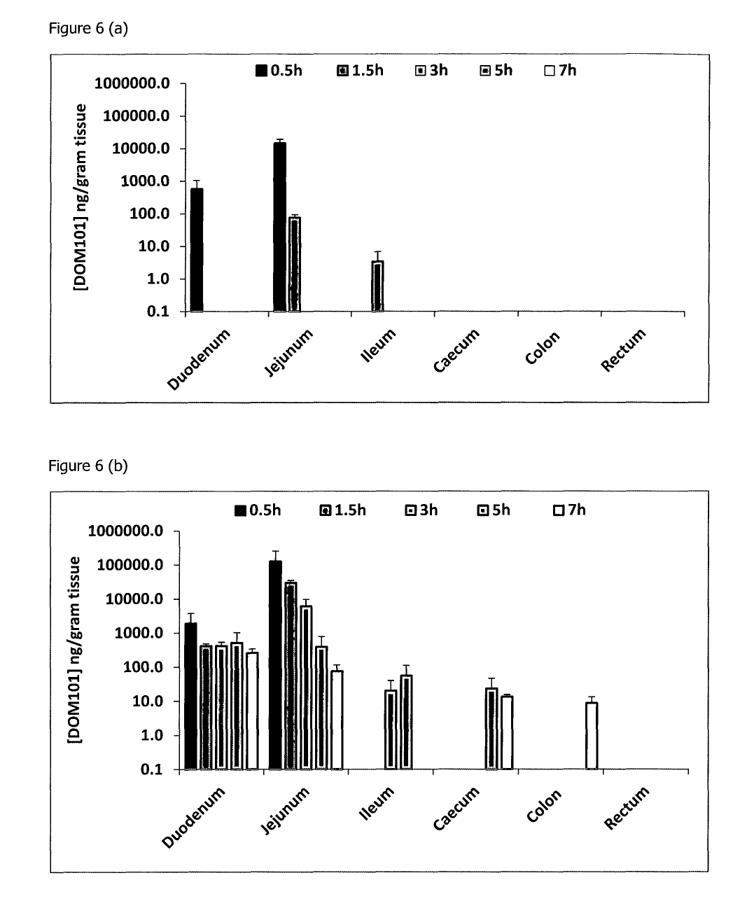

Results are expressed as nanograms per gram of tissue in FIG. 6.

In the absence of CM FIG. 6 (a), dAb™ was detectable in the duodenum only at 0.5 h, and only up to 1.5 h in the jejunum. The highest amount was detectable in the jejunum, and it was only detectable in the ileum in small amounts.

In the presence of CM FIG. 6 (b), dAb™ was detectable 7 h after dosing, throughout the GI tract. The dAb™ was only detectable in the ileum, caecum, colon and rectum at the later time-points. As before, the highest amount of dAb™ was recovered from the jejunum. Despite the likelihood of gut transit, dAb™ was also detectable in the duodenum and jejunum at 7 h, which suggested that dAb™ had penetrated the gut tissue. DOM101 was detectable at low levels in plasma (less than 0.1% of the total dose), after intra-duodenal dosing which confirmed that dAb™ can penetrate tissue—data not shown.

### Example 5

### Use of Camostat Mesylate to Stabilise the TNFR1 Specific dAb™ DOM101 (SEQ ID NO:6) Administered Directly into the Colon of Fasted Han Wistar Rats

Han Wistar rats were dosed with 1 mg DOM101 in the presence or absence of CM, to determine if camostat mesylate also preserved the dAb™ in the large intestinal tract. In brief, rats were anaesthetised by isoflurane anaesthetic, a midline abdominal incision made to facilitate location of the colon and 500 ul dose of the dose formulations injected directly into the colon. Following dosing, the abdominal incisions were loosely closed and the rats maintained under isoflurane anaesthesia and monitored for the duration of the experiment. In this Example, two doses of CM were studied—100 mg (as per Example 4) and 10 mg per animal.

Animals were culled at 0.5 and 3 hours (three animals per time-point). Blood samples were taken and the intestinal tract dissected out and divided into constituent parts as follows: caecum, colon (×2), rectum.

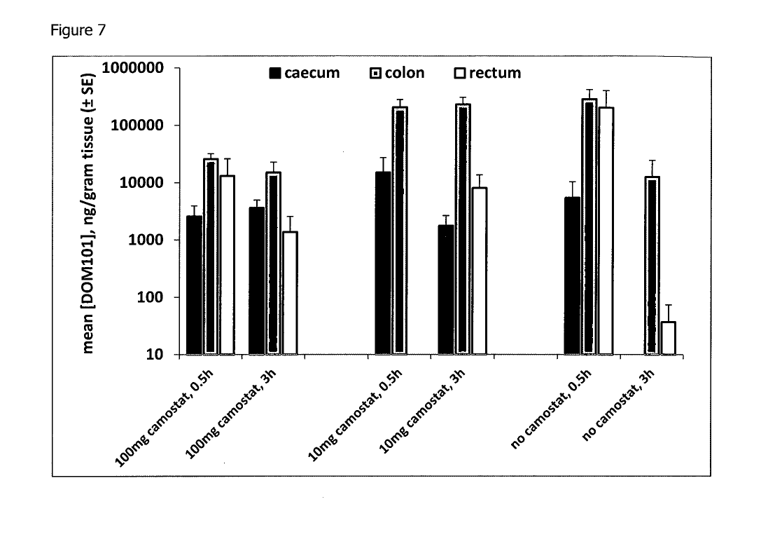

Samples were homogenised and screened as stated in Example 4. Results are expressed as nanograms per gram of tissue in FIG. 7.

High levels of dAb™ were detectable in the caecum, colon and rectum (except 10 mg camostat group) at 0.5 h, in the presence or absence of CM. There will be lower levels of digestive enzymes in the lower part of the GI tract which may explain this. The lack of dAb™ in the rectum at 0.5 h in the 10 mg camostat group is likely to be due to the higher wet weight of the caecum in these animals (data not shown)—dAb™ may therefore be retained in this section. However, by 3 h dAb™ levels in the absence of CM were substantially reduced, particularly in the caecum and rectum, compared with those observed in the two CM groups. The lower dose of CM (10 mg) appeared as effective as the higher dose at preserving dAb™ in the large GI tract.

### Summary of Examples 1-5

These Examples demonstrate that co-administration of camostat mesylate with a domain Antibody™ could be used as a novel platform for oral delivery of these molecules. Ten of the eleven dAbs™ studied in vitro were stabilised, to varying degrees, by addition of CM. When modelled in silico, a strong correlation was observed between half-life in the presence of CM and Tm, suggesting that the higher the Tm, the more amenable a dAbc™ is to stabilisation by CM. The comparison of two dAbs™ with identical predicted trypsin cleavage sites also shows the importance of Tm for intrinsic stability of dAbs™ in SIF.

The in vitro results are supported by the in vivo studies, where co-administration of camostat mesylate with DOM101 substantially increases the amount of dAb™ recoverable from the GI tract, whether delivered to the duodenum or the colon. Addition of CM to a formulation should allow topical delivery of dAbs™ to the duodenum or colon for the treatment of gastrointestinal conditions such as Crohn's Disease or ulcerative colitis.

SEQUENCE CONCORDANCE (all sequences
are amino acid sequences)

| SEQ ID NO | Identifier |
|---|---|
| 1 | DOM1 single variable domain |
| 2 | DOM2 single variable domain |
| 3 | DOM3 single variable domain |
| 4 | DOM6 single variable domain |
| 5 | DOM7 single variable domain |
| 6 | DOM101 single variable domain |
| 7 | human TNFα |
| 8 | human IL-23 |
| 9 | human LAG-3 |
| 10 | human IL-6 |
| 11 | human IL-13 |
| 12 | human IL-18 |
| 13 | human TSLP |
| 14 | human CD3D |
| 15 | human CD3E |
| 16 | human CD3G |
| 17 | human CD3Z |
| 18 | human TNFR1 |
| 19 | human CXCL2 |
| 20 | human CXCL5 |
| 21 | human GROA |
| 22 | human CXCL3 |

1. A composition comprising camostat mesylate and a single variable domain.

2-3. (canceled)

4. A composition as claimed in claim 1, wherein the single variable domain is an anti-target single variable domain, wherein the target is TNFα, IL-23, LAG-3, IL-6, IL-13, IL-18, TSLP, a CD3, a receptor of any one of the foregoing or an ELR receptor.

5. A composition as claimed in claim 1, wherein the single variable domain neutralises TNFα, IL-23, LAG-3, IL-6, IL-13, IL-18, TSLP or CD3.

6. A composition as claimed in claim 1, wherein the single variable domain has a transition midpoint (Tm) of greater than or equal to about 66° C.

7. A composition as claimed in claim 1, wherein the single variable domain to camostat mesylate ratio is about 1:0.1; 1:1; 1:10, 1:25, 1:50 or 1:100.

8. A composition as claimed in claim 1, wherein the composition is enterically coated.

9-12. (canceled)

13. A method of treating a gastrointestinal condition comprising the step of administering a composition as claimed in any one of claim 1 to a patient in need thereof.

14. A method of stabilising a single variable domain in a protease-rich solution comprising formulating the single variable domain in a composition comprising camostat mesylate prior to exposing the composition to a protease-rich solution.

15. A method as claimed in claim 14, wherein the single variable domain to camostat mesylate ratio is about 1:0.1; 1:1; 1:10, 1:25, 1:50 or 1:100.

16. (canceled)

17. A method as claimed in claim 15, wherein the protease-rich solution is a solution comprising trypsin, chymotrypsin and/or pancreatin.
